SEQUENCE LISTING (5 sequences)

SEQ ID NO: 1 - DNA, 199 bp, Homo sapiens
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggt

SEQ ID NO: 2 - protein, 1286 aa, Homo sapiens
MSHLVDPTSGDLPVRDIDAIPLVLPASKGKNMKTQPPLSRMNREELEDSFFRLREDHMLVKELSWKQQDEIKRLRTTLLRLTAAGRDLRVAEEAAPLSETARRGQKAGWRQRLSMHQRPQMHRLQGHFHCVGPASPRRAQPRVQVGHRQLHTAGAPVPEKPKRGPRDRLSYTAPPSFKEHATNENRGEVASKPSELVSGSNSIISFSSVISMAKPIGLCMPNSAHIMASNTMQVEEPPKSPEKMWPKDENFEQRSSLECAQKAAELRASIKEKVELIRLKKLLHERNASLVMTKAQLTEVQEAYETLLQKNQGILSAAHEALLKQVNELRAELKEESKKAVSLKSQLEDVSILQMTLKEFQERVEDLEKERKLLNDNYDKLLESMLDSSDSSSQPHWSNELIAEQLQQQVSQLQDQLDAELEDKRKVLLELSREKAQNEDLKLEVTNILQKHKQEVELLQNAATISQPPDRQSEPATHPAVLQENTQIEPSEPKNQEEKKLSQVLNELQVSHAETTLELEKTRDMLILQRKINVCYQEELEAMMTKADNDNRDHKEKLERLTRLLDLKNNRIKQLEGILRSHDLPTSEQLKDVAYGTRPLSLCLETLPAHGDEDKVDISLLHQGENLFELHIHQAFLTSAALAQAGDTQPTTFCTYSFYDFETHCTPLSVGPQPLYDFTSQYVMETDSLFLHYLQEASARLDIHQAMASEHSTLAAGWICFDRVLETVEKVHGLATLIGAGGEEFGVLEYWMRLRFPIKPSLQACNKRKKAQVYLSTDVLGGRKAQEEEFRSESWEPQNELWIEITKCCGLRSRWLGTQPSPYAVYRFFTFSDHDTAIIPASNNPYFRDQARFPVLVTSDLDHYLRREALSIHVFDDEDLEPGSYLGRARVPLLPLAKNESIKGDFNLTDPAEKPNGSIQVQLDWKFPYIPPESFLKPEAQTKGKDTKDSSKISSEEEKASFPSQDQMASPEVPIEAGQYRSKRKPPHGGERKEKEHQVVSYSRRKHGKRIGVQGKNRMEYLSLNILNGNTPEQVNYTEWKFSETNSFIGDGFKNQHEEEEMTLSHSALKQKEPLHPVNDKESSEQGSEVSEAQTTDSDDVIVPPMSQKYPKADSEKMCIEIVSLAFYPEAEVMSDENIKQVYVEYKFYDLPLSETETPVSLRKPRAGEEIHFHFSKVIDLDPQEQQGRRRFLFDMLNGQDPDQGHLKFTVVSDPLDEEKKECEEVGYAYLQLWQILESGRDILEQELDIVSPEDLATPIGRLKVSLQAAAVLHAIYKEMTEDLFS

SEQ ID NO: 3 - DNA, 3950 bp, Homo sapiens
atgtcacatctggtggaccctacatcaggagacttgccagttagagacatagatgctatacctctggtgctaccagcctcaaaaggtaagaatatgaaaactcaaccacccttgagcaggatgaaccgggaggaattggaggacagtttctttcgacttcgcgaagatcacatgttggtgaaggagctttcttggaagcaacaggatgagatcaaaaggctgaggaccaccttgctgcggttgaccgctgctggccgggacctgcgggtcgcggaggaggcggcgccgctctcggagaccgcaaggcgcgggcagaaggcgggatggcggcagcgcctctccatgcaccagcgcccccagatgcaccgactgcaagggcatttccactgcgtcggccctgccagcccccgccgcgcccagcctcgcgtccaagtgggacacagacagctccacacagccggtgcaccggtgccggagaaacccaagagggggccaagggacaggctgagctacacagcccctccatcgtttaaggagcatgcgacaaatgaaaacagaggtgaagtagccagtaaacccagtgaacttgtttctggttctaacagcataatttctttcagcagtgtcataagtatggctaaacccattggtctatgcatgcctaacagtgcccacatcatggccagcaataccatgcaagtggaagagccacccaagtctcctgagaaaatgtggcctaaagatgaaaattttgaacagagaagctcattggagtgtgctcagaaggctgcagagcttcgagcttccattaaagagaaggtagagctgattcgacttaagaagctcttacatgaaagaaatgcttcattggttatgacaaaagcacaattaacagaagttcaagaggcatacgaaaccttgctccagaagaatcagggaatcctgagtgcagcccatgaggccctcctcaagcaagtgaatgagctcagggcagagctgaaggaagaaagcaagaaggctgtgagcttgaagagccaactggaagatgtgtctatcttgcagatgactctgaaggagtttcaggagagagttgaagatttggaaaaagaacgaaaattgctgaatgacaattatgacaaactcttagaaagcatgctggacagcagtgacagctccagtcagccccactggagcaacgagctcatagcggaacagctacagcagcaagtctctcagctgcaggatcagctggatgctgagctggaggacaagagaaaagttttacttgagctgtccagggagaaagcccaaaatgaggatctgaagcttgaagtcaccaacatacttcagaagcataaacaggaagtagagctcctccaaaatgcagccacaatttcccaacctcctgacaggcaatctgaaccagccactcacccagctgtattgcaagagaacactcagatcgagccaagtgaacccaaaaaccaagaagaaaagaaactgtcccaggtgctaaatgagttgcaagtatcacacgcagagaccacattggaactagaaaagaccagggacatgcttattctgcagcgcaaaatcaacgtgtgttatcaggaggaactggaggcaatgatgacaaaagctgacaatgataatagagatcacaaagaaaagctggagaggttgactcgactactagacctcaagaataaccgtatcaagcagctggaaggtattttaagaagccatgaccttccaacatctgaacagctcaaagatgttgcttatggcacccgaccgttgtcgttatgtttggaaacactgccagcccatggagatgaggataaagtggatatttctctgctgcatcagggtgagaatctttttgaactgcacatccaccaggccttcctgacatctgccgccctagctcaggctggagatacccaacctaccactttctgcacctattccttctatgactttgaaacccactgtaccccattatctgtggggccacagcccctctatgacttcacctcccagtatgtgatggagacagattcgcttttcttacactaccttcaagaggcttcagcccggcttgacatacaccaggccatggccagtgaacacagcactcttgctgcaggatggatttgctttgacagggtgctagagactgtggagaaagtccatggcttggccacactgattggagctggtggagaagagttcggggttctagagtactggatgaggctgcgtttccccataaaacccagcctacaggcgtgcaataaacgaaagaaagcccaggtctacctgtcaaccgatgtgcttggaggccggaaggcccaggaagaggagttcagatcggagtcttgggaacctcagaacgagctgtggattgaaatcaccaagtgctgtggcctccggagtcgatggctgggaactcaacccagtccatatgctgtgtaccgcttcttcaccttttctgaccatgacactgccatcattccagccagtaacaacccctactttagagaccaggctcgattcccagtgcttgtgacctctgacctggaccattatctgagacgggaggccttgtctatacatgtttttgatgatgaagacttagagcctggctcgtatcttggccgagcccgagtgcctttactgcctcttgcaaaaaatgaatctatcaaaggtgattttaacctcactgaccctgcagagaaacccaacggatctattcaagtgcaactggattggaagtttccctacataccccctgagagcttcctgaaaccagaagctcagactaaggggaaggataccaaggacagttcaaagatctcatctgaagaggaaaaggcttcatttccttcccaggatcagatggcatctcctgaggttcccattgaagctggccagtatcgatctaagagaaaacctcctcatgggggagaaagaaaggagaaggagcaccaggttgtgagctactcaagaagaaaacatggcaaaagaataggtgttcaaggaaagaatagaatggagtatcttagccttaacatcttaaatggaaatacaccagagcaggtgaattacactgagtggaagttctcagagactaacagcttcataggtgatggctttaaaaatcagcacgaggaagaggaaatgacattatcccattcagcactgaaacagaaggaacctctacatcctgtaaatgacaaagaatcctctgaacaaggttctgaagtcagtgaagcacaaactaccgacagtgatgatgtcatagtgccacccatgtctcagaaatatcctaaggcagattcagagaagatgtgcattgaaattgtctccctggccttctacccagaggcagaagtgatgtctgatgagaacataaaacaggtgtatgtggagtacaaattctacgacctacccttgtcggagacagagactccagtgtccctaaggaagcctagggcaggagaagaaatccactttcactttagcaaggtaatagacctggacccacaggagcagcaaggccgaaggcggtttctgttcgacatgctgaatggacaagatcctgatcaaggacatttaaagtttacagtggtaagtgatcctctggatgaagaaaagaaagaatgtgaagaagtgggatatgcatatcttcaactgtggcagatcctggagtcaggaagagatattctagagcaagagctagacattgttagccctgaagatctggctaccccaataggaaggctgaaggtttcccttcaagcagctgctgtcctccatgctatttacaaggagatgactgaagatttgttttcatgaaggaacaagtgctattccaatctaaaagtctctgagggaaccatagtaaaaagtctcttataaagttagcttgctataacatgaaaaaa

SEQ ID NO: 4 - DNA, 29 bp, Artificial Sequence
  Primer
ggaattccccaggatcagatggcatctcc

SEQ ID NO: 5 - DNA, 29 bp, Artificial Sequence
  Primer
cccaagcttgcatggaggacagcagctgc

(19) United States
(12) Patent Application Publication
(10) Pub. No.: US 2014/0364488 A1
(43) Pub. Date: Dec. 11, 2014

(54) RPGRIP1 GENE THERAPY FOR LEBER CONGENITAL AMAUROSIS

(71) Applicant: Massachusetts Eye and Ear Infirmary, Boston, MA (US)

(72) Inventors: Basil Pawlyk, Hampton Falls, NH (US); Eliot L. Berson, Boston, MA (US); Tiansen Li, Clarksburg, MD (US); Michael A. Sandberg, Reading, MA (US)

(21) Appl. No.: 14/466,630

(22) Filed: Aug. 22, 2014

Related U.S. Application Data
(63) Continuation of application No. 14/122,163, now abandoned.
(60) Provisional application No. 61/493,186, filed on Jun. 3, 2011.

(51) Int. Cl. C12N 15/86

(52) U.S. Cl. CPC C12N 15/86; A61K 48/00
USPC 514/44 R; 604/506

(57) ABSTRACT

This invention relates to methods for treating subjects with vision loss due to advanced Leber Congenital Amaurosis (LCA), e.g., LCA6, which is due to loss-of-function mutations in the gene encoding the retinitis pigmentosa GTPase regulator interacting-protein-1 (RPGRIP1) protein.

### CLAIM OF PRIORITY

This application claims the benefit of U.S. Provisional Patent Application Ser. No. 61/493,186, filed on Jun. 3, 2011. The entire contents of the foregoing are hereby incorporated by reference.

### FEDERALLY SPONSORED RESEARCH OR DEVELOPMENT

This invention was made with Government support under Grant Nos. EY10581, P3OEY14104, and EY13600 awarded by the National Eye Institute of the National Institutes of Health. The Government has certain rights in the invention.

### TECHNICAL FIELD

This invention relates to methods for treating subjects with vision loss due to Leber Congenital Amaurosis (LCA), e.g., LCA6, which is due to loss-of-function mutations in the gene encoding the retinitis pigmentosa GTPase regulator-interacting protein-1 (RPGRIP1) protein.

### BACKGROUND

Retinitis Pigmentosa (RP) has a prevalence of about 1 in 4,000 affecting more than 1 million individuals worldwide (Berson, 1993). Patients with RP typically develop symptoms of night blindness during early adulthood followed by progressive loss of visual field and eventual blindness by 50-60 years of age (Berson, 1993). LCA is a more severe form of retinal degeneration with visual deficit in early childhood and loss of vision by the second and third decade of life (den Hollander et al., 2008; Fulton et al., 1996; Heher et al., 1992). Clinical findings indicate that both rod and cone photoreceptors are affected early in LCA patients. Mutations in at least 15 different genes are known to cause LCA (den Hollander et al., 2008; Koenekoop, 2004; Wang et al., 2009), one of which is the gene encoding the RPGRIP1 protein (Dryja et al., 2001; Gerber et al., 2001; Koenekoop, 2005). About 6% of all cases of LCA are caused by mutations in RPGRIP1 (den Hollander et al., 2008; Dryja et al., 2001; Gerber et al., 2001).

At present there is no effective treatment for LCA. Gene replacement therapy using delivery vectors derived from adeno-associated virus (AAV) has emerged as a promising potential therapy for retinal degeneration in recent years. Proof-of-principle experiments in animal models have been conducted for several forms of LCA, with varying degrees of success (Acland et al., 2001; Dejneka et al., 2004; Flannery et al., 1997; Narfstrom et al., 2003; Pang et al., 2006; Pawlyk et al., 2005; Sun et al., 2010; Tan et al., 2009). Phase I gene therapy trials in patients with LCA, targeting RPE65 gene defects in RPE cells, have been conducted (see, e.g., US2010/0272688). These clinical trials have yielded important preliminary outcomes indicating that this approach could be effective in restoring some degree of visual function (Bainbridge et al., 2008; Hauswirth et al., 2008; Maguire et al., 2008).

### SUMMARY

The present invention includes methods for treating subjects who have LCA caused by mutations in RPGRIP1. Subjects who can be treated by the present methods are those who have loss of visual function (i.e., impaired response on electroretinographic (ERG) testing), but retain some photoreceptor cells as determined by optical coherence tomography (OCT).

Thus, provided herein are methods for treating human subjects who have advanced Leber's Congenital Amaurosis (LCA) due to one or more loss-of-function mutations in the gene encoding the retinitis pigmentosa GTPase regulator interacting-protein-1 (RPGRIP1) protein. The methods include administering to the subject a nucleic acid comprising an adeno-associated viral vector comprising a human RPGRIP1 cDNA under the control of a human rhodopsin kinase (hRK) promoter.

In some embodiments, the subject has substantial visual impairment but retains substantially normal foveal thickness on optical coherence tomography.

In some embodiments, the visual impairment is demonstrated by the presence of hand motion or light perception vision.

In some embodiments, the visual impairment is demonstrated by the presence of an abnormal full-field ERGs (amplitude<1% of normal).

In some embodiments, the subject has a visual acuity of worse than 20/100. In some embodiments, the subject has a visual acuity of worse than 20/400.

In some embodiments, the adeno-associated viral vector is AAV-2, serotype-8 (AAV2/8). In some embodiments, the hRK promoter comprises SEQ ID NO:1. In some embodiments, the hRK promoter consists essentially of, or consists of, SEQ ID NO:1. In some embodiments, the human RPGRIP1 cDNA encodes a protein that is at least 95% identical to SEQ ID NO:2, e.g., is a sequence that is at least 95% identical to SEQ ID NO:3.

In some embodiments, the methods include administering the nucleic acid in a low dose of about 2×10¹⁰ vg/mL, a middle dose of about 2×10¹¹ vg/mL, or a high dose of about 2×10¹² vg/mL.

In some embodiments, the nucleic acid is administered into the subretinal space, e.g., via a micro injection cannula inserted into the subretinal space, temporal to the optic nerve and just above the major arcade vessels, so that fluid flow can be directed towards the macula.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram of the replacement gene construct in which the human RPGRIP1 cDNA was placed under the control of a human rhodopsin kinase (RK) promoter (AAV2/8-hRK-hRPGRIP1). The RK promoter is approx. 200 by in length and the hRPGRIP1 cDNA is approx. 4 kb. ITR, inverted terminal repeat; hRK, RK promoter; SV4OSD/SA, splice donor/acceptor sequences derived from the SV40 virus; wt pA+MZ, poly adenylation signal.

FIG. 1B is an image showing results of immunoblotting analysis with an anti-human RPGRIP1 antibody for expression of the transgenic protein following subretinal injection of AAV vectors. Subretinal delivery of the treatment vector led to expression of a 170-kDa protein (lane 1) in RPGRIP1^{-/-} mouse retina that co-migrated with the RPGRIP1 protein from human retina (lane 4). Endogenous RPGRIP1 in WT mouse retinas (lane 3) migrated at approx. 190 kDa (with a minor band at 170 kDa that may be either a degradative product or translation product from a minor transcript variant. Transducin a subunit (Ta) and a-acetylated tubulin were probed as loading controls.

FIG. 2A shows representative Dark-adapted (DA) and Light-adapted (LA) ERG waveforms from a pair of treated and control RPGRIP1^{-/-} eyes at 5 months of age. WT ERG waveforms are shown for comparison. The control eye had a profoundly reduced rod ERG and no detectable cone ERG at this age. The treated eye, however, had substantial rod and cone ERGs at this time point that are approximately at a third of WT values.

FIG. 2B shows representative light photomicrographs of the superior retina from the same pair of RPGRIP1^{-/-} eyes shown in 2A. The control eye had only 2 rows of photoreceptor cells remaining in the ONL at this age (5 months) with severely shortened (or absent) and disorganized inner/outer segments. In contrast, the treated eye had retained up to 5 rows of photoreceptor cells with longer and organized inner/outer segments.

FIG. 3 shows the sequences of human RPGRIP1 protein (SEQ ID NO:2) and nucleic acid (SEQ ID NO:3).

### DETAILED DESCRIPTION

The present methods include the use of a treatment vector that contains an RPGRIP1 gene cDNA (4 kb) and RK promoter (200 bp), both of human origin, packaged in an adeno-associated viral (AAV) vector, preferably the fast expressing AAV-2, serotype-8 (AAV2/8) delivery vector. The treatment vector is delivered into the eye of a subject who has been diagnosed with LCA due to mutations in RPGRIP1.

Retinitis Pigmentosa GTPase Regulator-interacting Protein-1 (RPGRIP1) RPGRIP1 is a protein that is normally localized to the photoreceptor connecting cilium, a thin bridge that links the inner and outer segments of the photoreceptor cell (Hong et al., J Biol Chem 2001; 276:12091-12099). It appears to be a stable component of the ciliary axoneme. A line of mutant mice lacking RPGRIP1 through targeted disruption of the RPGRIP1 gene has been described, and studies in these mice showed that one of the key functions of RPGRIP1 was to anchor RPGR in the connecting cilia (Zhao et al., Proc Natl Acad Sci U S A 2003; 100:3965-3970). In RPGRIP1^{-/-} mice, RPGR is mislocalized and no longer found at the connecting cilium. Thus, loss of RPGRIP1 would appear to encompass the loss of RPGR function as well. RPGRIP1 also performs additional function(s) at the connecting cilium, since mice lacking RPGRIP1 have a more severe retinal phenotype than mice lacking RPGR alone (Hong et al., Proc Natl Acad Sci USA 2000; 97:3649-54). RPGRIP1 mutant photoreceptors exhibit profound disruption of the outer segment structure and mislocalization of opsin proteins in rods and cones. Without wishing to be bound by theory, RPGRIP1 may be involved in photoreceptor disc morphogenesis. Both LCA patients (den Hollander et al., Prog Retin Eye Res 2008; 27:391-419; Fulton et al., Arch Ophthalmol 1996; 114:698-703; Heher et al., Ophthalmology 1992; 99:241-245) and mice lacking RPGRIP1 (Zhao et al., Proc Natl Acad Sci U S A 2003; 100:3965-3970; Pawlyk et al., Invest Ophthalmol Vis Sc 2005;46:3039-3045) have early onset rapid retinal degeneration.

The sequence of human RPGRIP1 can be found in GenBank at Accession No. NM_{—}020366.3 (nucleic acid) and NP 065099.3 (protein). The sequences of human RPGRIP1 can be at least 80%, e.g., 85%, 90%, 95%, or 100% identical to the full length of those sequences, e.g., to SEQ ID NOs. 2 and 3. The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

RK Promoter

In some embodiments of the methods described herein, a replacement gene construct is used in which a human RPGRIP1 cDNA is placed under the control of a human rhodopsin kinase (hRK) promoter (AAV2/8-hRK-hRPGRIP1). In some embodiments, the RK promoter is approx. 200 by in length (a short promoter derived from the rhodopsin kinase (RK) gene, which has been shown to drive cell-specific expression in rods and cones (Khani et al., 2007; Sun et al., 2010; Young et al., 2003)).

An exemplary hRK promoter sequence is −112/+87 (Khani et al., 2007):

(SEQ ID NO: 1)
GGGCCCCAGAAGCCTGGTGGTTGTTTGTCCTTCTCAGGGGAAAAGTGAGGC
GGCCCCTTGGAGGAAGGGGCCGGGCAGAATGATCTAATCGGATTCCAAGCA
GCTCAGGGGATTGTCTTTTTCTAGCACCTTCTTGCCACTCCTAAGCGT
CCTCCGTGACCCCGGCTGGGATTTAGCCTGGTGCTGTGTCAGCCCCGGT

Viral Delivery Vector

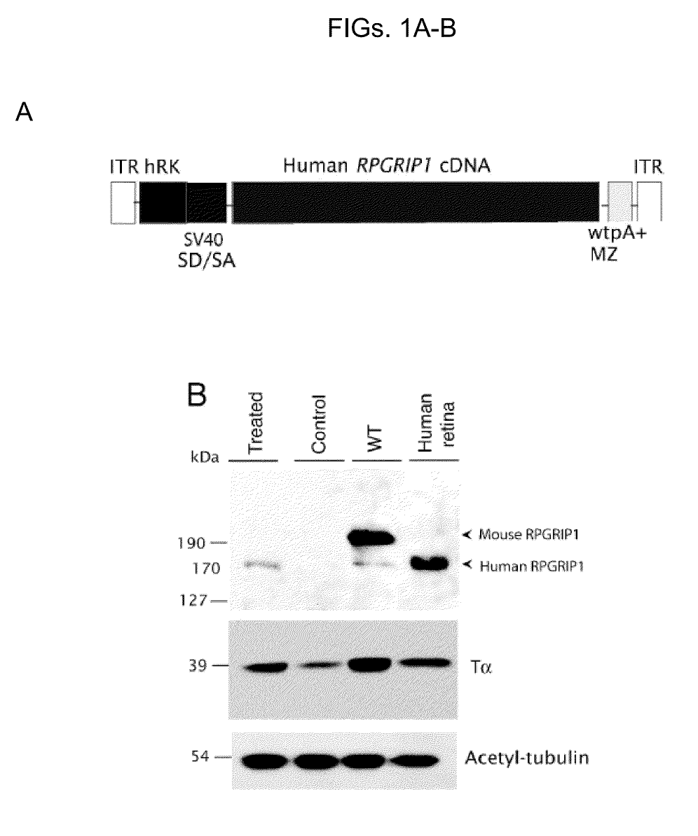

The hRPGRIP1 cDNA, as described above, is approx. 4 kb. This construct is packaged into a delivery vector. FIG. 1 A shows a schematic illustration of an exemplary construct, using an AAV2/8 vector.

Replacement genes (cDNA) can be administered in any effective carrier, e.g., any formulation or composition capable of effectively delivering the component gene to cells in vivo. Approaches include insertion of the gene into non-pathogenic, non-replicating viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign

DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immuno1.158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski ct al., J. Virol. 63:3822-3828 (1989); and McLaughlin ct al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

In preferred embodiments, the viral delivery vector is a recombinant AAV2/8 virus.

Prior to administration, the final product will undergo a series of ultrapurification steps to meet clinical grade criteria.

Subject Selection

Subjects are candidates for the present methods of treatment include those who have a diagnosis of LCA. Typical symptoms of LCA include: severe vision impairment from birth; nystagmus (involuntary jerky rhythmic eye movement); a normal-appearing eye upon visual examination (though there may be some pigmentation on the retina); extreme farsightedness; photophobia; a slow pupillary response to light; and markedly reduced ERGs. A diagnosis of LCA can be made, e.g., based on Lambert's criteria (Lambert et al., Sury Ophthalmol. 1989; 34(3):173-86).

The methods described herein can include identifying a subject, e.g., a child, adolescent, or young adult subject with LCA or who is suspected of having LCA (e.g., based on the presence of symptoms of LCA and no other obvious cause), and obtaining a sample comprising genomic DNA from the subject, detecting the presence of a mutation in RPGRIP1 using known molecular biological methods, and selecting a patient who has a mutation in RPGRIP1 that causes LCA. Detecting a mutation in RPGRIP1 can include detecting a specific known mutation, e.g., as described in Dryja et al., Am J Hum Genet. 2001; 68(5):1295-8, Gerber et al., Eur J Hum Genet. 2001; 9(8):561-71; or Lu et al., Hum Mol Genet. 2005 May 15;14(10):1327-40. Exemplary mutations include, e.g., a 1-bp deletion (T) at codon asp1176; a trp65-to-ter nonsense mutation; a 1-bp (T) insertion at codon g1n893; a 1-bp deletion (A) at codon lys342; an 3341A-G transition in exon 21 of the RPGRIP1 gene, resulting in an asp1114-to-gly (D1114G) substitution in the RPGR-interacting domain (RID) (rs17103671); a 3-bp deletion, or resulting in loss of glu1279 (de1E1279) located 8 residues upstream to the stop codon. Detecting a mutation in RPGRIP1 can also include sequencing all or part of the RGRIP1 gene in a subject, and comparing the sequence to a reference sequence (e.g., GenBank Accession No. NG_{—}008933.1), to detect a mutation. Frameshift mutations, truncation mutations, mutations that alter a conserved amino acid, or mutations that affect a regulatory (e.g., promoter) region can be considered to be mutations that can cause LCA; an alteration in function can be confirmed by expressing the mutant in vitro (e.g., in cultured cells) or in vivo (e.g., in a transgenic animal), and assaying, e.g., function or subcellular localization.

As demonstrated by optical coherence tomography, patients with LCA due to RPGRIP1 mutations can retain a substantial number of photoreceptors even when visual function has largely been lost as measured by visual field and ERGs. The methods described herein can include identifying subjects who have been diagnosed with LCA and have a mutation in RPGRIP1 that causes their LCA, and testing their visual ability and the presence of residual photoreceptors. Subjects, e.g., young adult subjects, who can be treated using the present methods have visual impairment as demonstrated by the presence of hand motion (while the subject can recognize a hand being waved, he or she cannot count the fingers on the hand) or light perception vision (see, e.g., Johnson, Deafness and Vision Disorders: Anatomy and Physiology, Assessment Procedures, Ocular Anomalies, and Educational Implications, Charles C. Thomas Publisher; 1999) Carlson, N; Kurtz, D.; Heath, D.; Hines, C. Clinical Procedures for Ocular Examination. Appleton & Lange; Norwalk, Conn. 1990), or abnormal full-field ERGs (amplitude <1% of normal), but a normal or near normal central foveal thickness on OCT (e.g., at least 75%, 80%, 90%, 95%, or 99% of normal thickness).

In some embodiments, the methods can also include identifying and treating subjects who have Cone-Rod Dystrophy 13 (Hameed et al., J Med Genet. 2003; 40(8):616-9), e.g., associated with a 2480G-T transversion in exon 16 of the RPGRIP1 gene, which changes codon 827 from CGC (arg) to CTC (leu) (rs28937883), or a 1639G-T substitution in exon 13 of the RPGRIP1 gene, which changed codon 547 from GCT (ala) to TCT (ser) (rs10151259).

### EXAMPLES

The invention is further described in the following examples, which do not limit the scope of the invention described in the claims.

### Example 1

### Expression of Human RPGRIP1 in Mouse Retina

The treatment vector contained an RPGRIP1 gene cDNA (4 kb) and RK promoter (200 bp), both of human origin, packaged in the fast expressing AAV2/8 delivery vector. Two weeks following subretinal injections with our prototype treatment vector (AAV2/8-hRK-hRPGRIP1) in 14-day-old RPGRIP1-/- mice, human RPGRIP1 protein was detected in the retina (see FIG. 1B). No RPGRIP1 protein was detected in eyes given a GFP control (AAV2/8-hRK-GFP). The human RPGRIP1 protein expressed from the replacement gene was identical in apparent molecular weight to that of the native RPGRIP1 from human donor retinal tissues. However, the human RPGRIP1 is predicted to have a molecular weight of 147 kDa, and the major mouse RPGRIP1 variant is predicted to have a molecular weight of 152 kDa values considerably smaller than those (170 kDa and 190 kDa) that was estimated based on motility on SDS-PAGE (polyacrylamide) gels. These differences can be reasonably explained by the high content of negatively charged amino acid resides (Glu) in these proteins, especially in mouse RPGRIP1. A higher acidic residue content is known to retard motility of the polypeptides on SDS-PAGE gels thus giving higher apparent molecular weight estimates (Graceffa et al., Arch Biochem Biophys 1992; 297:46-51; Korschen et al., Neuron 1995; 15:627-636; Lakoucheva et al., Protein Sci 2001; 10:1353-1362). For example, the RPGR protein exhibits a similar behavior because of high glutamic acid content, having a much higher apparent molecular weight than would be predicated from its sequence. The most important observation is that the recombinant human RPGRIP1 protein from treated mouse retinas was indistinguishable in apparent molecular weight from that of native human RPGRIP1 protein from donor retinas. Therefore it can be concluded that the human RPGRIP1 coding sequence used in the replacement gene construct is indeed the same form that is expressed endogenously in human photoreceptors. However, it appeared that the expression level of human RPGRIP1 protein was less in the treated retinas as compared to that expressed endogenously in WT mice.

Human RPGRIP1 protein localized correctly and is functional in mouse photoreceptors. This was demonstrated both by RPGR and opsin protein localization studies and by better photoreceptor function (ERG) and morphology following treatment in RPGRIP1^{-/-} mice. Human RPGRIP 1 localized correctly to the connecting cilia of mouse photoreceptors. Similar to endogenous mouse RPGRIP1 in WT mouse retina, human RPGRIP1 correctly localized to the photoreceptor connecting cilium just distal to the inner segment protein rootletin in the treated retina but not in the control retina. Expression of human RPGRIP1 led to the return of mouse RPGR at the connecting cilium, also just distal to rootletin.

### Example 2

### Human RPGRIP1 Partially Rescues Disease Phenotype in Knockout Mice Exhibiting Early Stage Disease

Introduction of human RPGRIP1 into 14-day-old RPGRIP1^{-/-} mice partially reversed the retinal disease phenotype in mice lacking RPGRIP1. At this age, the mice had only early stage disease, exhibiting substantially normal vision (as determined by ERGs) and photoreceptors (as determined by histology and immunofluorescence).

Subretinal injections were performed as follows. Mice were placed under general anesthesia with an intraperitoneal injection of ketamine (90 mg/kg)/xylazine (9 mg/kg). A 0.5% proparacaine solution was applied to the cornea as a topical anesthetic. Pupils were dilated with topical application of cyclopentolate and phenylephrine hydrochloride. Under an ophthalmic surgical microscope, a small incision was made through the cornea adjacent to the limbus using an 18-gauge needle. A 33-gauge blunt needle fitted to a Hamilton syringe was inserted through the incision while avoiding the lens and pushed through the retina. All injections were made subretinally in a location within the nasal quadrant of the retina. Each animal received 0.5-1 μl of AAV at 1×10¹² particles/ml. Treatment vector was typically given in the left eye (OS) and control vector was given in the fellow eye (OD), and they are referred to throughout this text as “treated” and “control”, respectively. Cohorts of mice (n=50) were injected at approximately postnatal day 14.

Histology and immunofluorescence was performed as follows. For both light microscopy and transmission electron microscopy, enucleated eyes were fixed for 10 minutes in 1% formaldehyde, 2.5% glutaraldehyde in 0.1 M sodium cacodylate buffer (pH7.5). Following removal of the anterior segments and lens, the eye cups were left in the same fixative at 4° C. overnight. Eye cups were washed with buffer, post-fixed in 2% osmium tetroxide, dehydrated through a graded alcohol series and embedded in Epon. Semi-thin sections (1 μm) were cut for light microscopic observations. For EM, ultrathin sections (70 nm) were stained in uranyl acetate and lead citrate before viewing on a JEOL 100CX electron microscope. For morphometric analyses of photoreceptor inner and outer segment (IS/OS) length and outer nuclear layer (ONL) thickness, measurements were made along the vertical meridian at 3 locations to each side of the optic nerve head separated by about 500 tm each. Measurements began at about 500 tm from the optic nerve head itself.

For immunofluorescence, eyes were enucleated, placed in fixative and their anterior segment and lens were removed. The fixative was 2% formaldehyde, 0.25% glutaraldehyde/PBS. Duration of fixation was typically 20 minutes. The fixed tissues were soaked in 30% sucrose/PBS for at least 2 hours, shock frozen and sectioned at 10-μm thickness in a cryostat. Sections were collected into PBS buffer and remained free floating for the duration of the immunostaining process. In some cases, eyes were unfixed and frozen sections were collected on glass slides. Sections were viewed and photographed on a laser scanning confocal microscope (model TCS SP2; Leica).

Antibodies used were anti-mouse RPGRIP1, anti-human RPGRIP1, anti-mouse RPGR (S1), anti-rootletin, anti-rhodopsin (rho 1D4; gift of Robert Molday) (Molday, 1988), green cone anti-opsin, and Hoechst 33342, nuclear dye stain. Rabbit anti-human RPGRIP1 was generated by Cocalico Biologicals, Inc., using amino acids 964-1274 from human RPGRIP1. Antigen was amplified by PCR, using the Origene clone as a template, and primers, sense: hRPGRIP-1S: GGAATTCCCCAGGATCAGATGGCATCTCC (SEQ ID NO:4); anti-sense: hRPGRIP-1R: CCCAAGCTTGCATGGAGGACAGCAGCTGC (SEQ ID NO:5). PCR product was inserted into pET-28 vector between EcoRI and HindIII sites, expressed in BL21, Codon+cells (Stratagcnc) and purified on His-tag binding column.

ERG recordings were performed as follows. Methods for recording dark- and light-adapted ERGs have been previously described (Khani et al., 2007; Sun et al., 2010). Briefly, mice were dark-adapted overnight, anesthetized, and had both pupils dilated. Rod dominated responses were elicited in the dark with 10-μs flashes of white light (1.37×10⁵ cd/m²) presented at intervals of 1 minute in a Ganzfeld dome. Light-adapted, cone responses were elicited in the presence of a 41 cd/m² rod-desensitizing white background with the same flashes (1.37×10⁵ cd/m²) presented at 1 Hz. ERGs were monitored simultaneously from both eyes, with signal averaging for cone responses.

Differences were seen at two weeks after vector delivery in both the quality and in the numbers of rods and cones comparing treated to control eyes. There was an improvement in rhodopsin and cone opsin localization patterns in the treated retinas. Rhodop sin normally localized in photoreceptor outer segments. In control (untreated) RPGRIP1^{-/-} retinas, the inner and outer segments were severely shortened and indistinguishable, and rhodopsin mislocalized to the inner segments and cell bodies. In the treated retinas, rod outer segments were elongated and rhodopsin was largely partitioned to the outer segments with the inner and outer segment layers clearly distinguishable from one another. Staining for green cone opsin showed more plentiful cone photoreceptors with better-preserved outer segments in the treated RPGRIP1^{-/-} mouse retina compared to the control. Some cone opsin mislocalization was still present in the treated retina compared to the WT. There was also a thicker outer nuclear layer in the treated retinas as compared to the untreated knockout controls.

Treatment significantly promoted photoreceptor survival and improved photoreceptor morphology in terms of inner/outer segment length and organization, which are important indicators of photoreceptor health. Retinal function was also greatly improved by treatment. Treated eyes had significantly larger rod and cone ERG amplitudes at all time points tested, and the rates of rod and cone ERG amplitude decline were markedly slowed (see Table 1). In fact, there was no significant loss of cone function between 3 and 5 months following treatment.

*TABLE 1
Rates of Change in ERG Amplitudes in Control and Treated Eyes

| | | | | Treated Eye | | |
|---|---|---|---|---|---|---|
| Com- | | Control Eye | | (logₑunit/ | | P-value |
| ponent | N | (logₑunit/mo.) | P-value | mo.) | P-value | OD-OS |
| Rod | 37 | −0.47 ± 0.03 | <0.0001 | −0.08 ± 0.02 | 0.0002 | <0.0001 |
| a-wave | | (−37%)** | | (−8%) | | |
| Rod | 37 | −0.25 ± 0.03 | <0.0001 | −0.08 ± 0.03 | 0.0038 | <0.0001 |
| b-wave | | (−22%) | | (−8%) | | |
| Cone | 16 | −0.29 ± 0.11 | 0.0212 | 0.20 ± 0.13 | n.s. | 0.0026 |
| b-wave | | (−25%) | | | | |

*Table 1 lists the mean logₑ monthly rates of change and corresponding levels of significance by ERG component for control and treated eyes based on mice tested between 2 and 5 months of age.. The last column gives the levels of significance for the difference in the rates for control (OD) and treated (OS) fellow eyes The mean rates of decline without treatment ranged from 22%/month for the rod b-wave to 37%/month for the rod a-wave and that, with treatment, these rates of decline fell to 8% far the rod a-wave and rod b-wave and to no detectable change for the cone b-wave - in each case a significant slowing of disease course.
**Parentheses include percent monthly rates of change for significant effects.

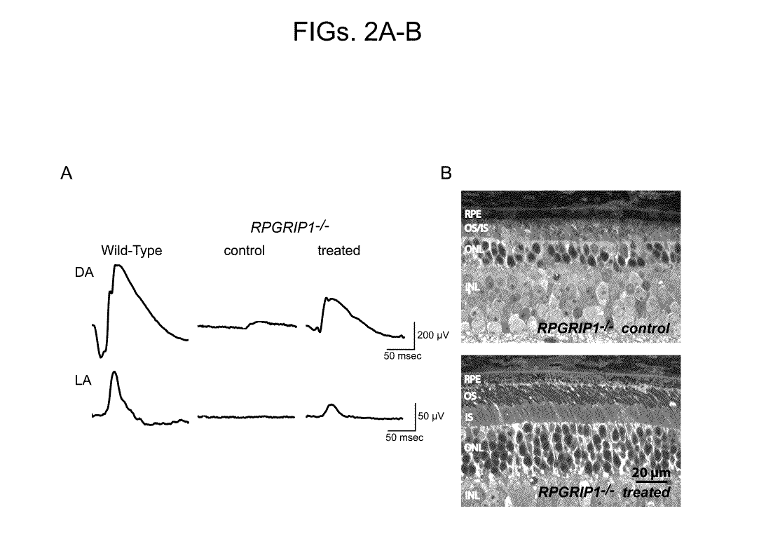

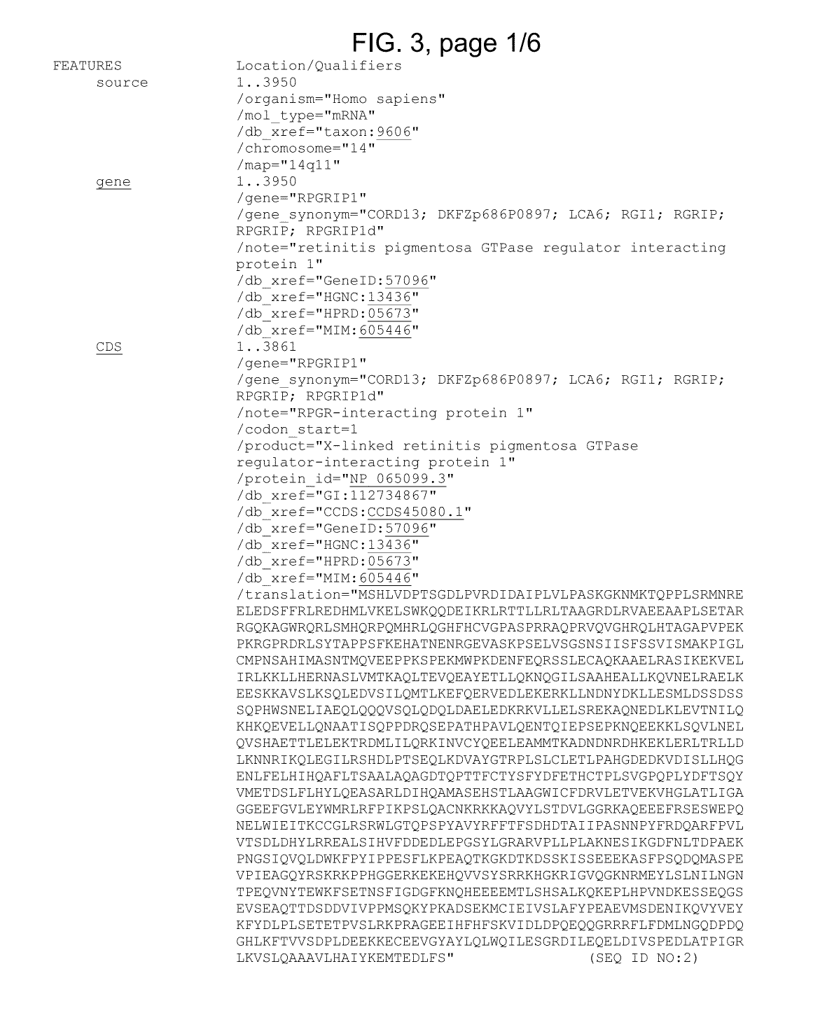

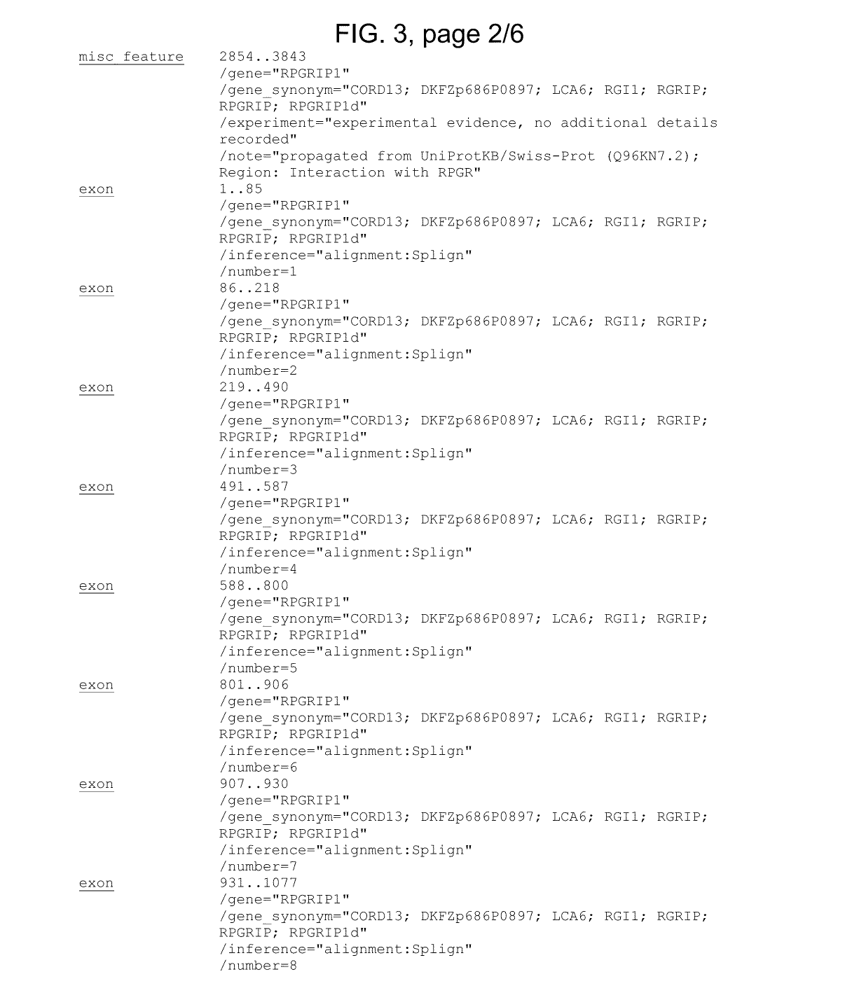

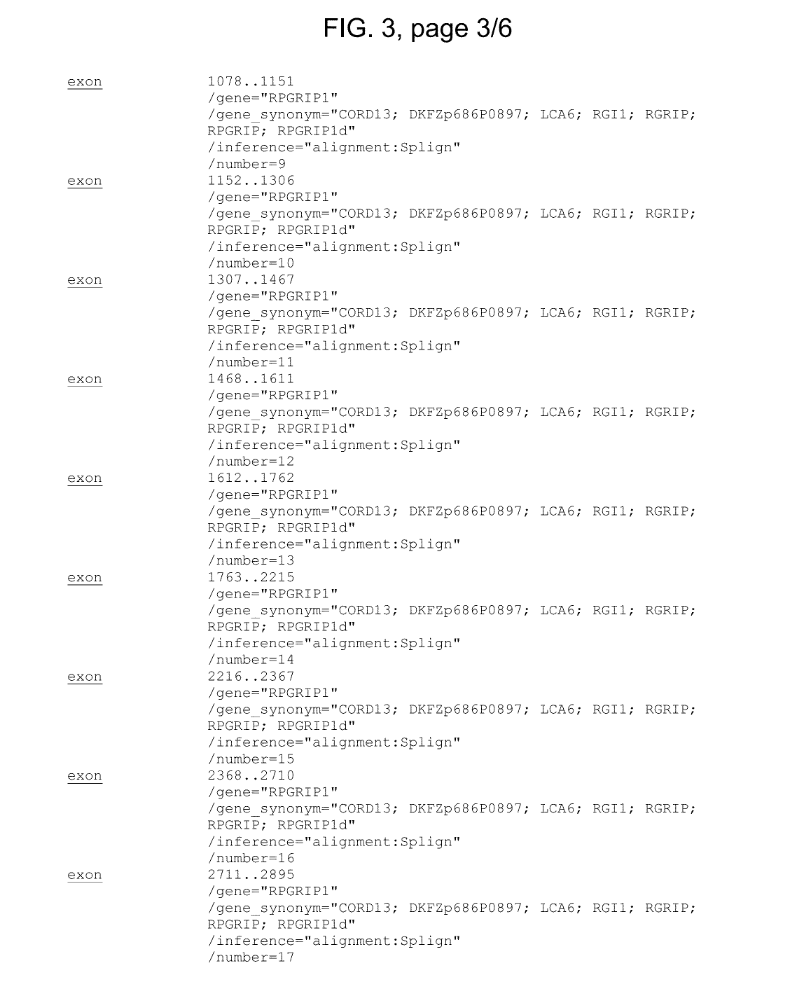

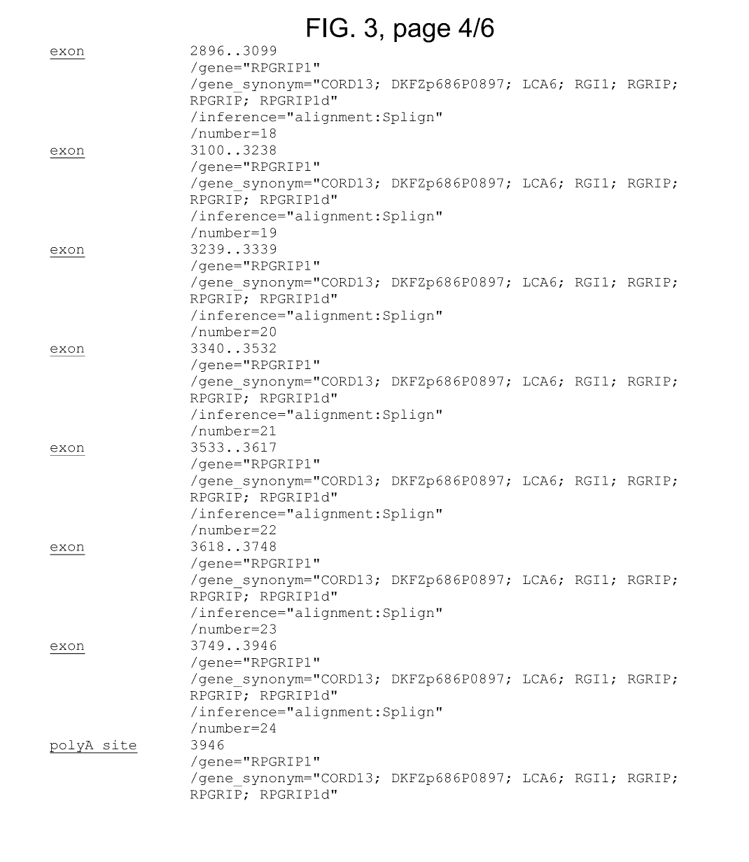

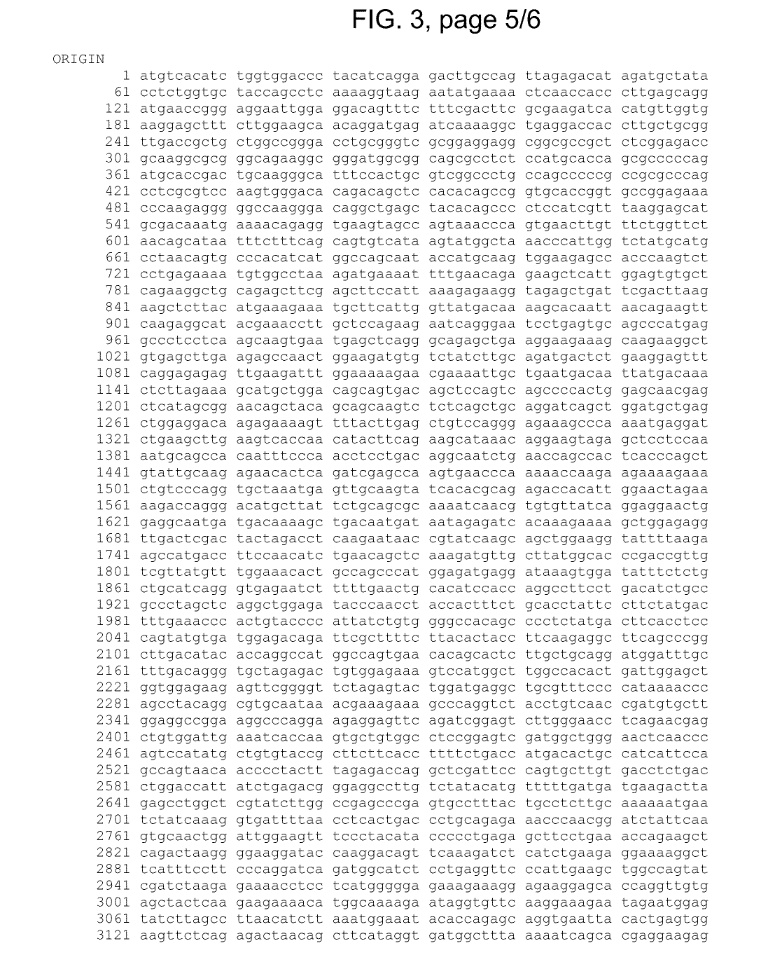

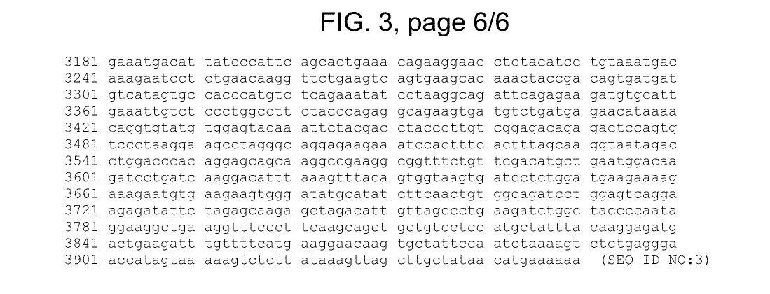

This suggests that cone function stabilized as a result of gene replacement therapy. By the final study time point (5 months), treated eyes had 200% larger rod ERGs and 400% larger cone ERGs than control eyes (see FIGS. 2A-B). These data confirm a therapeutic efficacy for this replacement gene construct in mouse photoreceptors and establish a prototype design for future clinical application in LCA patients with RPGRIP1 deficiency following safety and toxicity studies in animals.

Despite marked improvement in retinal function and morphology, however, rescue of the retinal disease phenotype was not complete. By comparison to WT mice, treated RPGRIP1^{-/-} mouse eyes had rod and cone ERG amplitudes that were on average approximately 30% of WT mouse values at 5 months of age—the end point of the study. Thus it would appear that the treatment did not fully reconstitute RPGRIP1 function in the recipient retinas, perhaps due to the reduced expression levels noted above. In addition, there is substantial divergence between human and mouse RGPRIP 1 sequences, with some regions of the protein bearing no homology between the two species. Another possibility is the existence of RPGRIP1 variants (Castagnet et al., 2003; Lu and Ferreira, 2005; Won et al., 2009). It remains unclear if and to what extent these variants are functionally significant in photoreceptors. The reported variants represent variant portions of the N-terminal RPGRIP1 and do not contain the RPGR interacting domain located at the C-terminus. Therefore, they are not expected to participate in the core function of RPGRIP 1, i.e., to anchor RPGR in the connecting cilia.

### Example 3

### Effect of Human RPGRIP1 on Disease Phenotype in Knockout Mice Exhibiting Advanced Disease

The experiments described in this example are performed to demonstrate that replacement gene therapy with human AAV-RPGRIP1 will rescue photoreceptors in a murine model of LCA with advanced disease.

RPGRIP1^{-/-} mice for study are bred from an existing colony and maintained under 12hr light/12 hr dark lighting cycle.

RPGRIP1^{-/-} mice are treated at 5 months of age (i.e., with an advanced stage of retinal degeneration). Subretinal injection techniques in mice are performed as previously described (Sun et al., Gene Ther 2010;1:117-3139; Pawlyk et al., Hum Gene Ther 2010; 21:993-1004). Following general anesthesia (80 mg/kg of sodium pentobarbital, IP) and pupilary dilation (2% phenylephrine hydrochloride and 0.2% cyclopentolate hydrochloride), a small incision is made in the cornea (adjacent to the limbus) with a hypodermic needle under a stereo dissecting microscope. A Hamilton syringe fitted with a 33-gauge blunt-ended needle is inserted around and past the lens until it meets resistance at the retina. Approximately 1 μl is injected, most of which will be retained subretinally causing a retinal detachment that can be monitored through the dissecting microscope. The superior ⅓ of the retina along the superior-inferior axis is targeted for injection, to facilitate subsequent analyses. Visualization of the injection process is aided by addition of fluorescein (100 mg/ml AK-FLUOR, Alcon, Inc.) to the vector suspensions at 0.1% by volume. 40 mice will receive therapeutic (treatment) AAV (AAV2/8-hRK-hRPGRIP1 @ 2×10′² vp/ml) in one eye and control AAV (AAV2/8-hRK-GFP) in the fellow eye. An additional 20 mice receive either therapeutic vector or control vector in both eyes for behavioral testing.

Visual Evoked Potentials (VEPs) are performed to verify that inner-retinal remodeling, seen in several murine models of retinal degeneration (Bulgakov et al., Invest Ophthalmol Vis Sci 2008;49 (ARVO Abstract)), has not specifically altered signal transmission through the proximal retina and more centrally. In a previous study of AIPL1-/- mice treated with replacement gene therapy VEP amplitudes were proportional to ERG amplitudes, indicating that any inner-retinal remodeling had not yet adversely affected signal transmission (Sun et al., Gene Ther 2010;1:117-3139).

Optomotor (behavioral) responses are measured using Optomotry (Cerebral Mechanics), a commercial system for objectively quantifying visual spatial resolution in awake rodents by monitoring their head movements (i.e., optokinetic tracking response) to laterally moving vertical gratings of varying spatial frequency generated by 4 computer monitors. This methodology has been used successfully in rodent models of retinal degeneration (Wang et al., Invest Ophthalmol Vis Sci 2008; 49:416-421) and should prove equally applicable for our mouse assessment. For these studies, WT mice are tested to obtain normative values.

Retinal histology and immunofluorescence are performed as follows. At 1,3, and 6 months following subretinal injections mice are euthanized and their eyes analyzed by histology to evaluate rod disease and by immunofluorescence to evaluate cone disease. Immunofluorescence is used to confirm the correct localization of human RPGRIP1 to the connecting cilia of photoreceptors. Methods will be as previously described (Sun et al., Gene Ther 2010;1:117-3139; Pawlyk et al., Hum Gene Ther 2010; 21:993-1004).

### Example 4

### Gene Replacement Therapy in Human LCA Patients

As demonstrated by optical coherence tomography, patients with LCA due to RPGRIP1 mutations can retain a substantial number of photoreceptors even when visual function has largely been lost as measured by visual field and ERGs. These patients can be treated by the methods described herein.

The eyes of patients with RPGRIP1 mutations are injected with 100 μL of a clinical grade human replacement gene construct (AAV2/8-hRK-hRPGRIP1) as described herein. Doses may range from low dose (e.g., about 2×10¹⁰ vg/mL) to middle dose (2×10¹¹ vg/mL) to high dose (2×10¹² vg/mL). This dose range and injection volume is similar to those used in recent gene replacement clinical trials for another form of LCA using AAV2/2 gene delivery vectors (Maguire et al., N Engl J Med 2008; 358:2240-8; Hauswirth et al., Hum Gene Ther 2008; 19:979-90; Bainbridge et al., N Engl J Med 2008;358:2231-9; Maguire et al., Lancet 2009; 374:1597-1605).

Patients with retinal degeneration (LCA) due to RPGRIP 1 gene mutations are recruited according to visual acuity and visual field criteria after signing informed consent for screening. Potentially eligible patients are screened with respect to visual acuities, visual fields, electroretinogram amplitudes, and optical coherence tomography profiles (OCTs). A complete ophthalmic examination is performed. Eligible patients have a visual acuity between 20/100 and hand motion vision in both eyes and no evidence of an epiretinal membrane by OCT. There must be evidence of some remaining photoreceptors by OCT.

Ocular function measurements, OCT, and the routine ophthalmic exam are performed the day before surgery. Surgery is performed using retrobulbar or peribulbar anesthetic block plus monitored intravenous sedation or general anesthesia, at the direction of a surgeon, administered by an anesthesiologist. A standard 3-port pars plana vitrectomy is performed to achieve a complete posterior vitreous detachment. For study agent delivery the surgeon places a micro injection cannula into the subretinal space, temporal to the optic nerve and just above the major arcade vessels, so that fluid flow can be directed towards the macula. As soon as positioning within the subretinal space is verified, a dosing assistant under instructions from the surgeon slowly injects the study agent into the subretinal space. The objective is for a broad, flat bleb directed towards a parafoveal region, within 1 disc diameter of the foveal center, where evidence of an outer nuclear layer was confirmed by spectral domain OCT. A 50% fluid (sterile saline)-air exchange is performed just prior to closing the sclerotomy sites with sutures. The retina is then examined with indirect ophthalmoscopy and scleral depression for any evidence of retinal breaks, detachment, or other problems. The postoperative intraocular pressure is measured as per the surgeon's usual protocol. Combined antibiotic and steroid ointment or drops are instilled in the eyes. The eyes are dressed with a sterile eye pad and a rigid shield. The operative sites are examined on the first postoperative day to assess for any untoward events or postoperative complications.

Patients are re-evaluated with respect to ocular function, OCT, and the ophthalmic exam following surgery.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a human subject who has advanced Leber's Congenital Amaurosis (LCA) due to a loss-of-function mutation in the gene encoding the retinitis pigmentosa GTPase regulator interacting-protein-1 (RPGRIP1) protein, the method comprising administering to the subject a nucleic acid comprising an adeno-associated viral vector comprising a human RPGRIP1 cDNA under the control of a human rhodopsin kinase (hRK) promoter.

2. The method of claim 1, wherein the subject has substantial visual impairment but retains substantially normal foveal thickness on optical coherence tomography.

3. The method of claim 2, wherein the visual impairment is demonstrated by the presence of hand motion or light perception vision.

4. The method of claim 2, wherein the visual impairment is demonstrated by the presence of an abnormal full-field ERGs (amplitude <1% of normal).

5. The method of claim 1, wherein the subject has a visual acuity of worse than 20/100.

6. The method of claim 1, wherein the subject has a visual acuity of worse than 20/400

7. The method of claim 1, wherein the adeno-associated viral vector is AAV-2, serotype-8 (AAV2/8).

8. The method of claim 1, wherein the hRK promoter comprises SEQ ID NO:1.

9. The method of claim 1, wherein the hRK promoter consists essentially of SEQ ID NO:1.

10. The method of claim 1, wherein the human RPGRIP1 cDNA encodes a protein that is at least 95% identical to SEQ ID NO:2.

11. The method of claim 1, comprising administering the nucleic acid in a low dose of about 2×10¹⁰ vg/mL, a middle dose of about 2×10¹¹ vg/mL, or a high dose of about 2×10¹² vg/mL.

12. The method of claim 1, wherein the nucleic acid is administered into the subretinal space.

13. The method of claim 11, wherein a micro injection cannula is inserted into the subretinal space, temporal to the optic nerve and just above the major arcade vessels, so that fluid flow can be directed towards the macula.
